(19) United States
(12) Patent Application Publication
(10) Pub. No.: US 2013/0129642 A1
(43) Pub. Date: May 23, 2013

(54) ORAL CARE COMPOSITIONS

(71) Applicants: Andrew Joiner, Bebington (GB); Carole Jane Philpotts, Bebington (GB)

(72) Inventors: Andrew Joiner, Bebington (GB); Carole Jane Philpotts, Bebington (GB)

(21) Appl. No.: 13/813,683

(22) PCT Filed: Jul. 28, 2011
(86) PCT No.: PCT/EP2011/063028

§ 371 Date: Feb. 1, 2013

(30) Foreign Application Priority Data
Aug. 6, 2010 (EP) 10172141.3

(51) Int. Cl. A61K 8/46; A61Q 11/00

(52) U.S. Cl. CPC A61K 8/466; A61Q 11/00
USPC 424/54

(57) ABSTRACT

The invention provides an oral care composition suitable for delivering a temporary whitening effect to the surface of teeth, the composition comprising: a continuous phase comprising water or polyhydric alcohol or a mixture thereof; a tooth surface whitening agent which is dispersed in the continuous phase, and a deposition aid for the tooth surface whitening agent; characterised in that the tooth surface whitening agent is a lake dye formed by fixing a dye onto a particulate inorganic substrate, in which the dye used to form the lake dye is a triarylmethane dye having a blue to green-blue colour with a hue angle in the CIELAB system ranging from 180 to 270 degrees, and in which the amount of lake dye is at least 0.015% by total weight lake dye based on the total weight of the composition.

### FIELD OF THE INVENTION

The present invention is concerned with oral care compositions. More particularly, the present invention is concerned with oral care compositions containing lake dyes.

### BACKGROUND OF THE INVENTION

The colour of the teeth is influenced by a combination of their intrinsic colour and the presence of any extrinsic stains that may form on the tooth surface. Extrinsic colour is linked with the adsorption of materials into the acquired pellicle on the surface of enamel, which ultimately cause staining. Factors that influence extrinsic stain formation include poor tooth brushing technique, smoking, dietary intake of coloured foods (e.g. red wine), subject age and the use of certain cationic agents such as chlorhexidine or metal salts like tin and iron.

Consumers have always had a strong desire for white teeth and many individuals are dissatisfied with their current tooth colour. This desire for whiter teeth has given rise to a growing trend in the increased use of tooth whitening products.

Current whitening toothpastes rely on optimised abrasive and chemical components to maximise stain removal and prevention. During brushing, abrasive particles become temporarily trapped between the toothbrush and the stained tooth surface and abrade away the stain. Chemical components may also be used, usually in conjunction with abrasive particles, and include calcium chelators, polymers, surfactants, enzymes, polymers and oxidising agents.

EP 1 935 395 describes a novel optical approach to tooth whitening. On brushing with the toothpaste described in this publication, a blue pigment (in particular blue covarine) is deposited onto the tooth surface, where it is able to change the optical effects of the tooth surface, and enhance the measurement and perception of tooth whiteness. This toothpaste is intended to produce a temporary tooth whitening effect that can be reapplied as frequently as desired, as it contains no harsh chemicals, but is not intended to produce any permanent changes to the colour of the teeth.

The present inventors have now found that lake dyes are particularly effective as temporary tooth whitening agents, and can produce superior temporary tooth whitening effects when used in a context similar to that described in EP 1 935 395.

U.S. Pat. No. 4,444,746 describes the use of lake dyes for imparting stable and effective coloration to visually clear dentifrices. However there is no suggestion that these materials might be effective for delivering a temporary whitening effect to the surface of teeth.

### SUMMARY OF THE INVENTION

The present invention provides an oral care composition suitable for delivering a temporary whitening effect to the surface of teeth, the composition comprising: a continuous phase comprising water or polyhydric alcohol or a mixture thereof; a tooth surface whitening agent which is dispersed in the continuous phase, and a deposition aid for the tooth surface whitening agent;

characterised in that the tooth surface whitening agent is a lake dye formed by fixing a dye onto a particulate inorganic substrate, in which the dye used to form the lake dye is a triarylmethane dye having a blue to green-blue colour with a hue angle in the CIELAB system ranging from 180 to 270 degrees, and in which the amount of lake dye is at least 0.015% by total weight lake dye based on the total weight of the composition.

### DETAILED DESCRIPTION OF THE INVENTION

Tooth Surface Whitening Agent

The composition of the invention comprises a tooth surface whitening agent which is dispersed in the continuous phase of the composition.

The tooth surface whitening agent is a lake dye formed by fixing a dye onto a particulate inorganic substrate, in which the dye used to form the lake dye is a triarylmethane dye having a blue to green-blue colour with a hue angle in the CIELAB system ranging from 180 to 270 degrees,

The colour to which the tooth surfaces are whitened depends on the colour of the dye used to form the lake dye. Preferred dyes used to form the lake dye have a blue to green-blue colour with a hue angle in the CIELAB system ranging from 180 to 200 degrees.

Particularly preferred dyes used to form the lake dye are water soluble. The term “water-soluble” in this particular context generally means that the dye has an aqueous solubility of at least 10 g/L at 25° C., most preferably at least 100 g/L at 25° C. (where the solubility is determined in un-buffered distilled water).

Aqueous solutions of particularly preferred dyes used to form the lake dye have a maximum absorbance value in the visible spectrum (λₘₐₓ) at a wavelength ranging from 550 to 650, more preferably from 600 to 650 nm.

Specific examples of preferred dyes used to form the lake dye include anionic triphenylmethane dyes, and especially diaminotriphenylmethane dyes containing from two to four sulphonate groups, such as those corresponding to general formula (I):

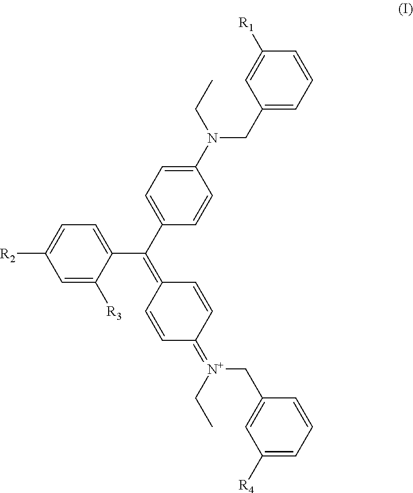

in which R¹ R²,R³ and R⁴ are monovalent radicals which are each independently selected from hydrogen (—H), hydroxyl
(—OH), halo (e.g. —Cl) and sulphonate (—SO₃⁻) groups, with the proviso that at least two of R¹ to R⁴ are sulphonate groups.

In a preferred example of a dye corresponding to general formula (I), R² is —H and R¹,R³, and R⁴ are sulphonate groups.

The particulate inorganic substrate onto which the dye is fixed is usually a colourless, inert metallic salt of synthetic or mineral origin. Examples of such materials include calcium carbonate, barium sulphate, kaolin (hydrated aluminium silicate), talc (hydrated magnesium silicate) and metal oxides such as titanium dioxide or zinc oxide. For optimum transparency the most preferred substrate is hydrated alumina, also known as alumina trihydrate or aluminium trihydroxide (Al(OH)₃). In a typical dye laking process, an aluminium salt is dissolved in water and an alkali is added to the solution in order to precipitate hydrated alumina. An aqueous solution of the dye is then added to a slurry of the precipitated particles, and a laking reagent (usually aluminium chloride) is added. This causes the dye to precipitate and adsorb onto the hydrated alumina particles, so forming the lake dye.

Typically the dye constitutes about 10 to 40% of the lake dye (by total weight dye based on the total weight of the lake dye).

Mixtures of any of the above described materials may also be used.

The most preferred lake dye for use in the invention

is formed by fixing a dye of formula (I) in which R² is —H and R¹,R³, and R⁴ are sulphonate groups onto particles of hydrated alumina. This material is commercially available for example as FD&C Blue No. 1 Aluminium Lake (C.I. 42090:2). The Colour Index number (C.I.) is taken from the Rowe Colour Index, 3rd edition, Society of Dyers and Colourists, Bradford, England, 1971.

The amount of lake dye (as defined above) in compositions of the invention suitably ranges from 0.015 to 1.0%, preferably from 0.015 to 0.5%, more preferably from 0.02 to 0.1% by total weight lake dye (as defined above) based on the total weight of the composition.

Deposition Aid

The composition of the invention comprises a deposition aid for the tooth surface whitening agent.

The term “deposition aid” in the context of this invention generally means a material which aids deposition of the tooth whitening agent from the continuous phase of the composition onto the surface of teeth during use of the composition. Use of the composition in the context of this invention typically involves application of the composition to the oral cavity, followed by brushing and/or rinsing).

Suitable deposition aids work by having affinity for both the lake dye (as defined above) and the surface of the teeth.

Preferred deposition aids are able to aid the deposition of the lake dye onto the teeth such that tooth surface whiteness is enhanced by at least 20% and more preferably by at least 100%, in comparison to the value obtained for teeth treated in an equivalent manner with a control formulation using the same amount of lake dye in the absence of the deposition aid.

A convenient measure of enhanced tooth surface whiteness is delta b* measured using a chromameter. A negative value of delta b* indicates a yellow to blue colour shift which has been shown to be one of the primary drivers of tooth surface whiteness as perceived by the consumer.

Accordingly, preferred deposition aids are able to aid the deposition of the lake dye onto the teeth such that the negative value of delta b* is augmented by at least 20% and more preferably by at least 100%, in comparison to the value obtained for teeth treated in an equivalent manner with a control formulation using the same amount of lake dye in the absence of the deposition aid.

A simple empirical model for establishing the effect of the deposition aid is as follows:

Polished hydroxyapatite discs are first placed in sterile human saliva for 2 hours to allow a pellicle to form. The discs are then rinsed in water and baseline colour measurements made (using, for example, a Minolta chromameter CR300). The discs are then brushed with either (a) a suspension of the lake dye in water; or (b) a suspension of the lake dye in water at the same concentration as in (a), together with the deposition aid. The brushing is best performed using a brushing machine. Following rinsing, the colour of the discs is then re-measured and the change in delta b* is recorded for both treatment (a) and treatment (b). From a comparison of these data, the effect of the deposition aid is readily seen.

Suitable deposition aids for use in the invention will generally dissolve or disperse in water at a temperature of 25° C.

Preferred deposition aids for use in the invention are water soluble. The term “water-soluble” in this particular context generally means that the deposition aid has an aqueous solubility of at least 10 g/L at 25° C., and more preferably at least 30 g/L at 25° C. (where the solubility is determined in un-buffered distilled water). It is particularly preferable that the deposition aid remains water soluble after drying, so that it can be re-dissolved. This prevents undesirable build up of the deposition aid on the teeth after repeated usage of the composition.

Suitable deposition aids for use in the invention include polymeric materials, preferably polymeric materials which are water soluble as defined above.

Polymeric materials for use as deposition aids in the invention may be naturally or synthetically-derived, and may be ionic or nonionic in nature.

Preferably such polymeric materials are high molecular weight. The term “high molecular weight” in this particular context generally means that the polymeric material has a molecular weight of at least 50,000, more preferably at least 500,000 g/mol. A suitable method to determine the molecular weight of such polymeric materials is gel permeation chromatography against a polyethylene glycol standard.

Specific examples of suitable classes of polymeric material for use as deposition aids in the invention include:

water-soluble, high molecular weight linear homopolymers of ethylene oxide characterised by the general formula H(OCH₂CH₂)ₙOH. These materials are generally termed polyethyleneoxides (or alternatively, polyoxyethylenes, or polyethylene glycols). In the general formula, n usually has an average value of at least 2000, preferably at least 50,000.

water-soluble, high molecular weight cellulose ethers such as methylcellulose, hydroxyethylcellulose, hydroxypropylcellulose, hydroxypropyl methylcellulose, hydroxybutyl methylcellulose, hydroxyethyl ethylcellulose, sodium carboxymethylcellulose, and sodium carboxymethyl hydroxyethylcellulose.

A preferred class of polymeric material for use as deposition aids in the invention includes water-soluble, high molecular weight polymers having anionic side groups along the polymer main chain.

Specific examples of such materials include poly(carboxylic acid) polymers. Poly (carboxylic acid) polymers are typically polymers which include —COON groups in their structure, or groups which are derived from —COOH groups such as salt, ester or anhydride groups.

For example, the poly(carboxylic acid) polymers may include:

—[C(R¹)(COOH)—]—

units in their structure, in which R¹ is selected from hydrogen, C₁₋₃ alkyl, C₁₋₃ alkoxy or C₁₋₃ hydroxyalkyl. Preferably R¹ is hydrogen.

A preferred type of poly (carboxylic acid) polymer includes adjacent:

—[C(R¹)(COOH)—]—

units in its structure (where R¹ is as defined above), for example polymers based on maleic acid, which typically include:

—{—CH(COOH)—CH(COOH)—]—

units, and/or salts or esters of such units, or such units in anhydride form in which —COOH groups on adjacent carbon atoms are cyclised to form a ring system.

For example, the poly(carboxylic acid) polymers may comprise units with pairs of carboxylic acid groups on adjacent polymer chain carbon atoms, for example polymers comprising:

—[—C(R¹)(R²)—C(R³)(R⁴)—C(R⁵)(COOH)—C(R⁶)(COOH)—]—

units in its structure (and/or salts or esters of such units, or such units in anhydride form in which —COOH groups on adjacent carbon atoms are cyclised to form a ring system); in which R¹,R²,R³,R⁴,R⁵ and R⁶ are each independently selected from hydrogen, C₁₋₃ alkyl or C₁₋₃ alkoxy. Preferably R¹ and R² are hydrogen, R³ is hydrogen and R⁴ is methoxy and R⁵ and R⁶ are hydrogen.

Such a poly(carboxylic acid) polymer may be described as the polymer based on a copolymer of methyl vinyl ether and maleic anhydride, and is commercially available for example under the trade name Gantrez®.

A particularly preferred example of such a polymer comprises:

—[—CH₂—CH(OCH₃)—CH(COOH)—CH(COOH)—]—

units in its structure, in which the —COOH groups are in free acid form. Such polymers may be linear or cross-linked. More preferably the polymer is linear. Polymers of this type are commercially available for example under the trade name Gantrez® S. Most preferably such a polymer has a molecular weight of at least 500,000 g/mol (e.g. Gantrez® S-96), ideally at least 1,000,000 g/mol (e.g. Gantrez® S-97).

Alternative polymers which may be used comprise the units as described above in anhydride form, i.e. in which the two adjacent —COOH groups are cyclised to form a ring system. Such polymers are commercially available under the tradename Gantrez® AN, e.g. Gantrez® AN-119, Gantrez® AN-903, Gantrez® AN-139 and Gantrez® AN-169.

Other alternative polymers which may be used comprise the units as described above in partial salt form, for example in which some of the free —COOH groups are converted into a metal salt of a Group I or Group II metal such as either sodium or calcium, or a mixed sodium-calcium salt. Such polymers are commercially available under the tradename Gantrez® MS, e.g. Gantrez® MS-955.

Other alternative polymers which may be used comprise the units as described above in partial ester form, for example in which some of the free —COON groups are esterified with C₁₋₆ alkyl, e.g. ethyl or n-butyl. Such polymers are commercially available under the tradename Gantrez® ES, e.g. Gantrez® ES-225 or Gantrez® ES-425.

Mixtures of any of the above described materials may also be used.

The amount of deposition aid (as defined above) in compositions of the invention suitably ranges from 0.001 to 5.0%, preferably from 0.005 to 4.0%, more preferably from 0.01 to 2.0% by total weight deposition aid (as defined above) based on the total weight of the composition.

Product Form

The composition of the invention comprises a continuous phase comprising water or polyhydric alcohol or a mixture thereof.

Examples of suitable product forms for compositions of the invention include dentifrices, mouthwashes, chewing gums and lozenges.

A preferred type of product form in the context of the present invention is a dentifrice. The term “dentifrice” generally denotes formulations which are used to clean the surfaces of the oral cavity. The dentifrice is an oral composition that is not intentionally swallowed for purposes of systemic administration of therapeutic agents, but is applied to the oral cavity, used to treat the oral cavity and then expectorated. Typically the dentifrice is used in conjunction with a cleaning implement such as a toothbrush, usually by applying it to the bristles of the toothbrush and then brushing the accessible surfaces of the oral cavity. Preferably the dentifrice is in the form of a paste or a gel (or a combination thereof).

A dentifrice composition according to the invention will usually contain a liquid continuous phase in an amount of from 40 to 99% by weight based on the total weight of the dentifrice. Such a liquid continuous phase will typically comprise a mixture of water and polyhydric alcohol in various relative amounts, with the amount of water generally ranging from 10 to 45% by weight (based on the total weight of the dentifrice) and the amount of polyhydric alcohol generally ranging from 30 to 70% by weight (based on the total weight of the dentifrice). Typical polyhydric alcohols include humectants such as glycerol, sorbitol, polyethylene glycol, polypropylene glycol, propylene glycol, xylitol (and other edible polyhydric alcohols), hydrogenated partially hydrolyzed polysaccharides and mixtures thereof.

A dentifrice composition according to the invention will generally contain further ingredients to enhance performance and/or consumer acceptability such as abrasive cleaning agent, binder or thickening agent, and surfactant.

For example, a dentifrice will usually comprise an abrasive cleaning agent in an amount of from 3 to 75% by weight based on the total weight of the dentifrice. Suitable abrasive cleaning agents include silica xerogels, hydrogels and aerogels and precipitated particulate silicas; calcium carbonate, dicalcium phosphate, tricalcium phosphate, calcined alumina, sodium and potassium metaphosphate, sodium and potassium pyrophosphates, sodium trimetaphosphate, sodium hexametaphosphate, particulate hydroxyapatite and mixtures thereof.

Furthermore, the dentifrice will usually contain a binder or thickening agent in an amount of from 0.5 to 10% by weight based on the total weight of the dentifrice. Suitable binders or thickening agents include carboxyvinyl polymers (such as polyacrylic acids cross-linked with polyallyl sucrose or polyallyl pentaerythritol), hydroxyethyl cellulose, hydroxypropyl cellulose, water soluble salts of cellulose ethers (such as sodium carboxymethyl cellulose and sodium carboxymethyl hydroxyethyl cellulose), natural gums (such as carrageenan, gum karaya, guar gum, xanthan gum, gum arabic, and gum tragacanth), finely divided silicas, hectorites, colloidal magnesium aluminium silicates and mixtures thereof.

Furthermore, the dentifrice will usually contain a surfactant in an amount of from 0.2 to 5% by weight based on the total weight of the dentifrice. Suitable surfactants include anionic surfactants, such as the sodium, magnesium, ammonium or ethanolamine salts of C₈ to C₁₈ alkyl sulphates (for example sodium lauryl sulphate), C₈ to C₁₈ alkyl sulphosuccinates (for example dioctyl sodium sulphosuccinate), C₈ to C₁₈ alkyl sulphoacetates (such as sodium lauryl sulphoacetate), C₈ to C₁₈ alkyl sarcosinates (such as sodium lauryl sarcosinate), C₈ to C₁₈ alkyl phosphates (which can optionally comprise up to 10 ethylene oxide and/or propylene oxide units) and sulphated monoglycerides. Other suitable surfactants include nonionic surfactants, such as optionally polyethoxylated fatty acid sorbitan esters, ethoxylated fatty acids, esters of polyethylene glycol, ethoxylates of fatty acid monoglycerides and diglycerides, and ethylene oxide/propylene oxide block polymers. Other suitable surfactants include amphoteric surfactants, such as betaines or sulphobetaines. Mixtures of any of the above described materials may also be used.

Another preferred type of product form in the context of the present invention is a mouthwash. The term “mouthwash” generally denotes liquid formulations which are used to rinse the surfaces of the oral cavity and provide the user with a sensation of oral cleanliness and refreshment. The mouthwash is an oral composition that is not intentionally swallowed for purposes of systemic administration of therapeutic agents, but is applied to the oral cavity, used to treat the oral cavity and then expectorated.

A mouthwash composition according to the invention will usually contain an aqueous continuous phase. The amount of water generally ranges from 70 to 99% by weight based on the total weight of the mouthwash.

A mouthwash composition according to the invention will generally contain further ingredients to enhance performance and/or consumer acceptability, such as the humectants and surfactants mentioned above for dentifrices. The amount of humectant generally ranges from 5 to 20% by weight based on the total weight of the mouthwash and the amount of surfactant generally ranges from 0.1 to 5% by weight based on the total weight of the mouthwash.

Compositions of the present invention (such as in particular dentifrices or mouthwashes) may also contain further optional ingredients customary in the art such as fluoride ion sources, anticalculus agents, buffers, flavouring agents, sweetening agents, colouring agents, opacifying agents, preservatives, antisensitivity agents and antimicrobial agents.

The invention is further illustrated with reference to the following, non-limiting Examples.

### EXAMPLE

### Formulations

Paste compositions having the ingredients shown in Table 1 below were used to prepare test pastes.

All ingredients are expressed by weight percent of the total formulation, and as level of active ingredient.

TABLE 1

| | Blue | Blue | Sheath |
|---|---|---|---|
| Ingredient | Formulation A | Formulation 1 | Formulation |
| Water | 7.955 | 6.497 | 9.33 |
| Sorbitol | 66 | 66 | 66 |
| PEG 32 | 2 | 2 | 2 |
| Sodium fluoride | 0.32 | 0.32 | 0.32 |
| Sodium saccharin | 0.20 | 0.20 | 0.2 |
| Trisodium phosphate | 1.1 | 1.1 | 0 |
| Gantrez ® S-97 | 1.0 | 1.0 | 0 |
| Thickening silica | 6 | 6 | 9 |
| Abrasive silica | 11 | 11 | 9.5 |
| Cellulose gum | 0.7 | 0.7 | 0.5 |
| Sodium lauryl sulphate | 1.8 | 1.8 | 1.8 |
| Flavour | 1.3 | 1.3 | 1.3 |
| Mica | 0 | 0 | 0.05 |
| Blue Covarine | 0.625 | 0 | 0 |
| (C.I.74160); | | | |
| 40% a.i. | | | |
| FD&C Blue No.1 | 0 | 2.083 | 0 |
| Aluminium Lake (C.I. | | | |
| 42090:2); 12% a.i. | | | |
| Total | 100 | 100 | 100 |

Test pastes were prepared, as follows:

Comparative Example A (not according to the invention): 10% w/w of Blue Formulation A and 90% w/w of Sheath Formulation.

Example 1 (according to the invention): 10% w/w of Blue Formulation 1 and 90% w/w of Sheath Formulation.

Each paste was made into an aqueous slurry (paste:water, 1:2), to give a final concentration of blue colorant in each case of 0.025% w/w, and evaluated for whitening performance as follows:

### Method

Hydroxyapatite (HA) discs were polished using P1200 grit silicon carbide paper and thoroughly rinsed with water. The discs were immersed in sterilised whole human saliva for a minimum of two hours to allow a pellicle to form. The HA discs were removed from the saliva, rinsed with water and the baseline colour of the discs were measured with a colorimeter in the CIELAB mode. The HA discs were then placed in a brushing machine and 10 ml of the test slurry added per disc. The discs were brushed for 1 minute under a brushing load of 375 g. After brushing the HA discs were rinsed in water and the colour of the discs re-measured. Changes in the colour co-ordinates L*, a* and b* and whiteness index (WIO) were calculated. The WIO index is an index which has been optimised specifically for the evaluation of whiteness in teeth (as described in Journal of Dentistry, Volume 36, Supplement 1, 2008, pages 2 to 7).

### Results

The changes in L*, a*, b* and whiteness (WIO) are shown in Table 2 below. For changes in WIO, the product differences were of statistical significance (p<0.05, Tukey-Kramer).

TABLE 2
Mean changes in colour parameters (s.d.)

| Formulation | Delta L* | Delta a* | Delta b* | Delta WIO |
|---|---|---|---|---|
| Comparative | −2.40 (0.60) | −1.37 (0.31) | −6.74 (0.84) | 11.09 (1.72) |
| Example A | | | | |
| Example 1 | −2.75 (0.62) | −6.17 (0.91) | −7.00 (0.53) | 17.32 (1.21) |

The results demonstrate the superior temporary tooth whitening effect obtained with the formulation of Example 1, compared with Comparative Example A which uses the preferred blue pigment of EP 1 935 395.

1. An oral care composition suitable for delivering a temporary whitening effect to the surface of teeth, the composition comprising:
a continuous phase comprising water or polyhydric alcohol or a mixture thereof;
a tooth surface whitening agent which is dispersed in the continuous phase, and
a deposition aid for the tooth surface whitening agent;
characterised in that the tooth surface whitening agent is a lake dye formed by fixing a dye onto a particulate inorganic substrate, in which the dye used to form the lake dye is a triarylmethane dye having a blue to green-blue colour with a hue angle in the CIELAB system ranging from 180 to 270 degrees, and in which the amount of lake dye is at least 0.015% by total weight lake dye based on the total weight of the composition.

2. An oral care composition according to claim 1, in which the dye used to form the lake dye has a hue angle in the CIELAB system ranging from 180 to 200 degrees.

3. An oral care composition according to claim 1, in which the particulate inorganic substrate onto which the dye is fixed is hydrated alumina.

4. An oral care composition according to claim 1, in which the deposition aid is a poly(carboxylic acid) polymer having a molecular weight of at least 50,000 g/mol and an aqueous solubility of at least 10 g/L at 25° C.

5. An oral care composition according to claim 1, which is in the form of a dentifrice or a mouthwash.

6. (canceled)
